(19) United States
(12) Patent Application Publication
(10) Pub. No.: US 2014/0357550 A1
(43) Pub. Date: Dec. 4, 2014

(54) Pharmaceutical Composition for Treatment of Acute Toxic Conditions

(71) Applicant: OOO "NTpharma", Moscow (RU)

(72) Inventors: Maksim M. SHMAROV, Moscow (RU); Raisa I. YAKUBOVSKAYA, Moscow (RU); Rustam R. ATAULLAKHANOV, Moscow (RU)

(21) Appl. No.: 14/459,975

(22) Filed: Aug. 14, 2014

Related U.S. Application Data
(63) Continuation of application No. PCT/RU2012/000363, filed on May 11, 2012.

(30) Foreign Application Priority Data
Feb. 16, 2012 (RU) 2012105304

(51) Int. Cl. A61K 38/40

(52) U.S. Cl. CPC A61K 38/40; A61K 48/00
USPC 514/2.5

(57) ABSTRACT

A pharmaceutical composition for treatment of acute toxic conditions relates to the field of medicine, particularly to nanotechnology and toxicology, and can be used for prophylaxis and therapy of various etiologies of toxic states, including the acute ones. The claimed pharmaceutical composition for treatment of acute toxic conditions contains protein—the human lactoferrin—and further comprises of non-replicating nanoparticles with inset of human lactoferrin gene and formulating buffer. The dose of the claimed pharmaceutical composition is 3 ml. The dose of the claimed pharmaceutical composition comprises: human lactoferrin—from 50 to 100 mg; non-replicating nanoparticles—7×10¹¹ virus particle (v.p.); formulating buffer—rest, ml. At the same time, the donor human milk lactoferrin or any human lactoferrin is used as the human lactoferrin.

### RELATED APPLICATIONS

This Application is a Continuation application of International Application PCT/RU2012/000363, filed on May 11, 2012, which in turn claims priority to Russian Patent Applications No. RU 2012105304, filed Feb. 16, 2012, both of which are incorporated herein by reference in their entirety.

### FIELD OF THE INVENTION

The invention relates to medicine, in particular to nanotechnology and toxicology, and can be used for prophylaxis and therapy of various etiologies of toxic states, including the acute ones.

### BACKGROUND OF THE INVENTION

A method of treatment of postoperative complications by using medicines containing protein antioxidants (Russian Patent No. 2199337) is well known. In this patent, the treatment of postoperative complications with the phenomena of multiple organ failure is carried out by the combined administration of a patient of a drug containing human lactoferrin and a drug containing human ceruloplasmin; these drugs containing human lactoferrin and human ceruloplasmin are administered systemically—daily—intravenously in isotonic glucose solution or sodium chloride. Also, a drug containing the human ceruloplasmin is administered intravenously daily systemically and a solution preparation, containing human lactoferrin, is used for daily washing of purulent wounds, cavities, and/or to irrigate the airways.

Lactoferrin is a metals-binding single chain protein. It is generally known that this protein has a number of natural therapeutic properties, including bactericidal and bacteriostatic activity, and it is involved in the regulation of cellular and humoral immune responses, inflammatory and other processes.

The disadvantage is that although the drug is acting immediately after administration, but only during the first 24 hours, and then it is eliminated from the body, which is a significant disadvantage in the treatment, as it is necessary to re-administer the drug often, as a part of the introduction of therapy.

An antibacterial, antioxidant, detoxifying, immune modulatory and anti-carcinogenic formulation, according to Russian Patent No. 2165769 is known, which contains human lactoferrin as a primary active agent and pharmaceutically acceptable additives; wherein the formulation comprises in wt. percentage: human lactoferrin—10.0-90.0, pharmaceutically acceptable additives—the rest. The claimed medicament can be in the form of a solution for internal administration, in the form of a solution for intra-cavity or intra-vesicle instillation, in the form of a solution for oral administration, in the form of a solution for treating of wound surfaces, in the form of eye drops, in the form of a solution for intranasal application, in the form of an ointment, in the form of a bolus for oral administration, in the form of suppositories for rectal or intra-vaginal use, in the form of a tablet.

This mentioned Patent is selected by the Authors as a prototype.

The disadvantage is also the fact that although the claimed drug is acting immediately after injection, but only during the first 24 hours, and then it is eliminated from the body; this is a significant disadvantage in therapy because of the need for frequent administration of the drug.

Common shortcomings of these compositions and formulations, and methods of treatment are:

1) difficulties in achieving a stable therapeutic effect in their application, due to the presence as active components of isolated and purified proteins—lactoferrin, which rapidly routes from the body of the patient for any excreted way of administration;

2) in order to maintain therapeutically effective concentrations in the body there is a necessity of multiple administration of pharmaceutical compositions containing lactoferrin;

3) costs of large amounts of the drug, medical instruments and medical staff time to achieve the desired outcome of treatment;

4) for the compositions and formulations which include lactoferrin obtainable from human milk—uniqueness of this raw material and its deficiency—severely limits scaling production and, accordingly, the possibility of its application in the required amounts in accordance with medical indications.

Also the recombinant lactoferrin produced in various systems, including viral vectors carrying the gene of human lactoferrin is known; it is similar to the native lactoferrin in its physical, biochemical and biological properties (Gonzalez-Chavez S. A., et al., Lactoferrin: structure, function and applications, Int. J. of Antimicrobial Agents, 2009, No. 33, hh. 301-308).

An effective method of treatment of induced mammary tumors in mice by administering to tumor tissue of a recombinant adenovirus carrying a human lactoferrin gene is known. This composition had a prolonged action and was administered once every two weeks (Wang J., et al., Inhibition of tumor growth by recombinant adenovirus containing human lactoferrin through inducing tumor cell apoptosis in mice bearing EMT6 breast cancer, Arch. Pharm. Res., 2011, No. 34 (6), pp. 987-995).

Disadvantage of this composition is as follows. The expression of the target protein lactoferrin, using adenovirus vector construction, in the body does not begin with the introduction of the drug, but in a few hours, which is a subsequent disadvantage in relation to the treatment of acute toxic effects; although the level of lactoferrin is building and is lasting for a long time after just a single injection, which is advantageous for the treatment of chronic toxicosis but cannot be used for the treatment of acute toxic effects.

### SUMMARY OF THE INVENTION

Technical aim of the presented invention is directed to the building of a pharmaceutical composition with sustained and rapidly commencing antitoxic effect, based on nanostructures, producing human lactoferrin directly in the body and that is suitable for treatment of acute toxic states of various origins.

This problem is solved by the following means: the pharmaceutical composition for the treatment of acute toxic conditions containing protein—the human lactoferrin—further comprises a non-replicating nanoparticles with insert of human lactoferrin gene, and a formulating buffer. At this, the drug dose is 3 ml. The dose of the claimed pharmaceutical composition comprises of:
human lactoferrin from 50 to 100 mg;
non-replicating nanoparticles—7×10¹¹ virus particle (v.p.);
formulating buffer—rest, ml.

At the same time, lactoferrin of the donor human milk or any other human lactoferrin is used as human lactoferrin.

Technical solution of the application is realized due to the fact that the combination of properties of native human lactoferrin derived from a donor human milk and human lactoferrin expressed from non-replicating nanoparticles based on the genome of Adenovirus Serotype 5 with insertion of exogenous DNA, comprising a gene encoding a protein (the human lactoferrin) is used in one pharmaceutical composition that allows for treatment of acute toxic states of various etiologies with a single dose of the drug. The composition also contains a formulating buffer as a pharmaceutically acceptable additive. That is what provides for effective work of lactoferrin from almost since its introduction and prolong its therapeutic concentration for 28-30 days, without implementation of additional injections.

Technical, medical and economic results when carrying out the claimed invention are achieved due to the fact that, just as in the known drug based on human lactoferrin extracted from human milk, a major therapeutic agent in the proposed drug is human lactoferrin, the level of which in the body after administration of an invented pharmaceutical composition is provided by native lactoferrin and after that the level is provided by recombinant lactoferrin produced by non-replicating nanoparticles.

A pharmaceutical composition, according to the invention, is made in the dosage form of:
human lactoferrin from 50 to 100 mg;
non-replicating nanoparticles—7×10¹¹ virus particle (v.p.);
formulating buffer—rest, ml;
and it is used as a solution for intravenous administration.

The pharmaceutical composition serves as a starting material for preparation of various dosage forms, the use of which is determined by the pathogenesis of toxicosis. The inventive pharmaceutical composition based on the native human lactoferrin and non-replicating nanoparticles with insert of a gene, encoding human lactoferrin, has passed preclinical and clinical trials of specific (therapeutic) efficiency and systemic toxicity, which showed the harmlessness of the composition and its activity as detoxifying therapeutic agent for various toxic conditions, particularly acute toxicity, which is illustrated by the following examples.

### BRIEF DESCRIPTION OF THE DRAWINGS

FIG. 1 shows the pharm-cinematic curve characterizing the concentration of human lactoferrin in the serum of rats after a single intravenous introduction of a pharmaceutical composition in a dose of non-replicating nanoparticles 4.3×10¹¹ v.p./m² and native lactoferrin 10 mg/kg. In the axis of abscissa time in days is shown, in the axis of ordinate concentration of lactoferrin in serum, mkg/ml is shown.

FIG. 2 presents data of duration of thiopental sleep of animals inoculated with CCl₄. The bars indicate the duration of sleep in groups of animals treated with:
CCl₄;
0.9% solution of sodium chloride;
native human lactoferrin;
composition containing only non-replicating nanoparticles with insert of lactoferrin gene;
the pharmaceutical composition.

In the axis of ordinate—time of thiopental sleep in min. is shown.

### DETAILED DESCRIPTION OF THE PREFERRED EMBODIMENTS

The below examples represent:
construction of non-replicating nanoparticles and building of their capacity to the required amount;
creation of a pharmaceutical composition;
an evidence of rapid appearance of lactoferrin in blood and of a prolonged action of the pharmaceutical composition;
a proof of an anti-toxic effect of the pharmaceutical composition created according to the claimed invention.

### Example 1

### Construction of Non-Replicating Nanoparticle Based on the Genome of Adenovirus Serotype 5 with Insert of Human Lactoferrin Gene

Construction of the non-replicating nanoparticle based on the genome of Adenovirus Serotype 5 (size: 70-80 nm) with inset of human lactoferrin gene was based on the recombinant plasmid pJM17 (Mc Grory W J, A simple technique for the rescue of early region I mutations into infectious Adenovirus Serotype 5, Virology, No. 163 (2), 1988, p. 614), with a deletion of the adenoviral genome in the El region. All subsequent cloning manipulations were performed using well-known laboratory techniques (e.g., Sambrook, J., et al, Methods of genetic engineering: molecular cloning, World, Moscow, 1984, pp. 205-224, 387-420). Cloning was performed by homologous recombination in cell culture and its gist is as follows. Artificially synthesized cDNA of human lactoferrin gene in selected restriction sites was over-cloned into well-known shuttle-plasmid pRcCMV (Invitrogen, San Diego, Calif., No. V75020). Further on, for mutual transformation of the obtained plasmid pRcCMV-Lf and vector plasmid pJM17, they were used for and transfection of cells 293 (e.g., CLS, Germany, No. 300192) using the method of calcium phosphate precipitation (Graham F. L., et al., A new technique for the assay of infectivity of Adenovirus Serotype 5 DNA, Virology, 1973, No. 52 (2), pp. 456-467). The result was a non-replicating nanoparticles containing an expression cassette with CMV-promoter, the human lactoferrin gene and a polyadenylation signal. Plaques of the recombinant particles were formed by cell culture a few days after transfection; they were collected with a Pasteur pipette, the resulting material was propagated on line 293 cells to obtain a titer of 3×1010 v.p. (virus particle)/ml (108 PFU/ml).

Predetermined content of non-replicating nanoparticles and native lactoferrin in a pharmaceutical composition was defined by pharm-kinetics and antitoxic effect in Examples 4 and 5.

For obtaining of such pharmaceutical composition, the cell suspension accumulated in the previous step, comprising of a non-replicating nanoparticles titer 3×1010 virus particle/ml, was used for further increasing of titers of non-replicating nanoparticle and preparation of finished pharmaceutical composition containing at least 2.33×1011 virus particle/ml (corresponding with an activity of at least 6.7×108 PFU/ml) and 50-100 mkg of native lactoferrin (e.g., specified in Russian Patent No. 2165769) in 3 ml of composition.

Thus, further on, for production of required titers of non-replicating nanoparticles, a wave bioreactor with 4500 ml of suspension of a permissive cell culture 293 was inoculated with cell culture of 500 ml containing non-replicating nanoparticles with titer 3×1010 v.p./ml.

The cell mass was cultured to full capacity of non-replicating nanoparticles inside cells and achievement of their content up to 6×1010 v.p./ml (activity of 2×108 PFU/ml) for approximately 48 hours. Upon achievement of necessary content of nanoparticles the cell mass was fed for cleaning, a process which consisted of several stages:

Precipitation of cell mass was made by centrifugation. The coming for treatment suspension had at least 1014 v.p. for 5 liters (was assessed by mass spectrometry, 1 a.u. (absorbance unit=1012 v.p.). Centrifugation was performed at 6000-gyro mode for 15 min, liquid supernatant was decanted and the remaining solid portion, containing cells and non-replicating nanoparticles, was fed to further purification steps.

Removing of non-replicating nanoparticles from the cell culture was carried out by a four-stage destruction of cells by refreezing-thawing. Also, a buffer solution of pH 8.0 was prepared: 5 mMTrisHCl, 0.075 MNaCl, 1 mMMgCl2, 5% sucrose, 1% polysorbate-80. Sediment, obtained in the previous step was re-suspended in 70 ml of buffer (content ratio—x71). Volume of the solution was 80 ml. Freezing was performed in liquid nitrogen for 2 hours, thawing—in a water bath (at +37° C.) avoiding overheating.

To facilitate further removal of genomic cellular DNA an additional processing with nuclease was performed. To achieve this, benzonase was added until concentration in solution up to 150 units/ml and the solution was placed for a soft stirring with usage of a magnetic stirrer for 3 hours at room temperature (21-23° C.).

Separation of non-replicating nanoparticles from destroyed cells was performed by centrifugation at 9000-gyro for 10 minutes. Supernatant containing non-replicating nanoparticles was then collected.

Further purification was carried out by ultrafiltration. For this purpose, the resultant supernatant was diluted with buffer (50mM TrisHCl pH 7.5, 1M NaCl, 2 mM MgCl2, 5% sucrose, pH 7.5) to a volume of no less than 200 ml, stirred with a magnetic stirrer. During the filtration, volume of the circulating solution (retentate) were constantly made up to the initial one (200 ml).

Further purification was carried out by anion-exchange chromatography. The retentate was applied to a column (AxiChrom 70/300 with volume of 400 ml) containing anion-exchange sorbent Q Sepharose Virus Licenced. Non-replicating nanoparticles thus concentrated on the column, while impurities did not concentrate and were eluted with buffer “A”. After removing of impurities non-replicating nanoparticles were desorbed by washing with “B”. Chromatography conditions were as follows: flow 193 ml/min, buffer “A” (40 mM TrisHCl, 0.27 M NaCl, 2 mM MgCl2, 5% sucrose, 0.1% polysorbate 80, pH 7.5), conductivity of ˜28-30 mS/cm; buffer “B” (40mM TrisHCl, 0.5M NaCl, 2 mM MgCl2, 5% sucrose, 0.1% polysorbate 80, pH 7.5), conductivity of ˜50 mS/cm. The eluate of 200 ml in volume was sent to the next step of processing.

Exclusion chromatography. The eluate extracted in the previous step was applied to the column (AxiChrom 100/300 with volume of 800 ml) containing sorbent Q Sepharose 4 FastFlow. Macromolecular substances not included in the pores of the sorbent were eluted with the first peak (which includes non-replicating nanoparticles) impurities were eluted after the peak with non-replicating nanoparticles. Chromatographic conditions were as follows: flow 130 ml/min, buffer (10 mMTrisHCl, 75 mMNaCl, 1 mMMgCl2, 5% sucrose, 0.05% polysorbate 80, pH 8.0). To the extracted eluate (80 ml)—was added ethanol to a concentration of 0.5% and ethylenediaminetetraacetic acid (EDTA) to a concentration of 100 mcM—was sent to the subsequent stage.

Normal filtration. Sterilization of the resulting formulation was carried out through a system of filters with pore size of 22 mcM. Final volume of the substance at this stage was 80 ml and contained non-replicating nanoparticles in titer of 1×1012 ph.par./ml. It was diluted with formulating buffer (e.g., 10 mMTrisHCl, 75 mMNaCl, 1 mMMgCl2, 5% sucrose, 0.05% polysorbate 80, 0.5% ethanol, 100 microns of EDTA, pH 8.0) to reach a content of 2.33×1011 v.p./ml and was sterilized by normal filtration.

### Example 2

### Process of Preparation of Pharmaceutical Composition

To obtain the final pharmaceutical solution of the claimed composition prepared in the previous stage, the drug was mixed with the concentrate of native human lactoferrin extracted from human milk (Russian Patent No. 2165769), located in the buffer used for formulation of the drug from non-replicating nanoparticles in the previous stage (e.g., 10 mM Tris, 75 mM sodium chloride, 5% sucrose, 0.05% Tween-80, 1 mM Magnesium chloride, 0.5% ethanol, 100 microns of EDTA, pH 8.0). Miscible volumes of solution of non-replicating nanoparticle concentrate and lactoferrin were of such nature, that the predetermined content of non-replicating nanoparticles was as a result finally obtained—2.33×10¹¹ v.p./ml (which corresponds to the activity of the drug in 6.7×10⁸ PFU/ml) as well as from 50 mg to 100 mg of native lactoferrin in 3 ml of the composition.

### Example 3

### Stability of the Pharmaceutical Composition

Pharmaceutical composition obtained in Example 2 was evaluated for formulation stability.

For this, visual assessment of a sample, under close observation, was conducted for 3 minutes. Visual assessment showed good miscibility of components of the drug and absence of clots.

Table 1 shows the effect of the components of the pharmaceutical composition on the stability of non-replicating nanoparticles. The evaluation was conducted after exposure to a pharmaceutical composition for zero, 30 and 60 minutes, with a further assessment of titles of non-replicating nanoparticles according to the standard procedures.

TABLE 1

| | Time of exposition, min | | |
|---|---|---|---|
| Pharmaceutical composition or control | 0 | 30 | 60 |
| substance | Titles, v.p./ml | | |
| Pharmaceutical composition | 3 × 10⁸ | 3 × 10⁸ | 3 × 10⁸ |
| Culture medium (control substance) | 3 × 10⁸ | 3 × 10⁸ | 3 × 10⁸ |

Data in Table 1 indicate conservation titers of non-replicating nanoparticles upon exposure of the pharmaceutical composition from 0 minutes to 1 hour, which corresponds to their safety in control substance.

Thus, these results show the stability of the resulting pharmaceutical composition.

### Example 4

### Selection of Doses of Native Human Lactoferrin and Non-Replicating Nanoparticles Expressing Human Lactoferrin

Ability to use the pharmaceutical composition for treatment of acute toxicity was assessed for its pharmacokinetics state when administered to laboratory animals (rats) intravenously in a volume dose containing non-replicating nanoparticles expressing human lactoferrin equal to 4.3×10¹¹ v.p./m² and a dose of native lactoferrin equal to 10 mg/kg.

Evaluation was carried out by the presence and elimination of the target protein—human lactoferrin in the organs. The figure shows the pharmacokinetic curve representing the concentration of human lactoferrin in the serum of mice.

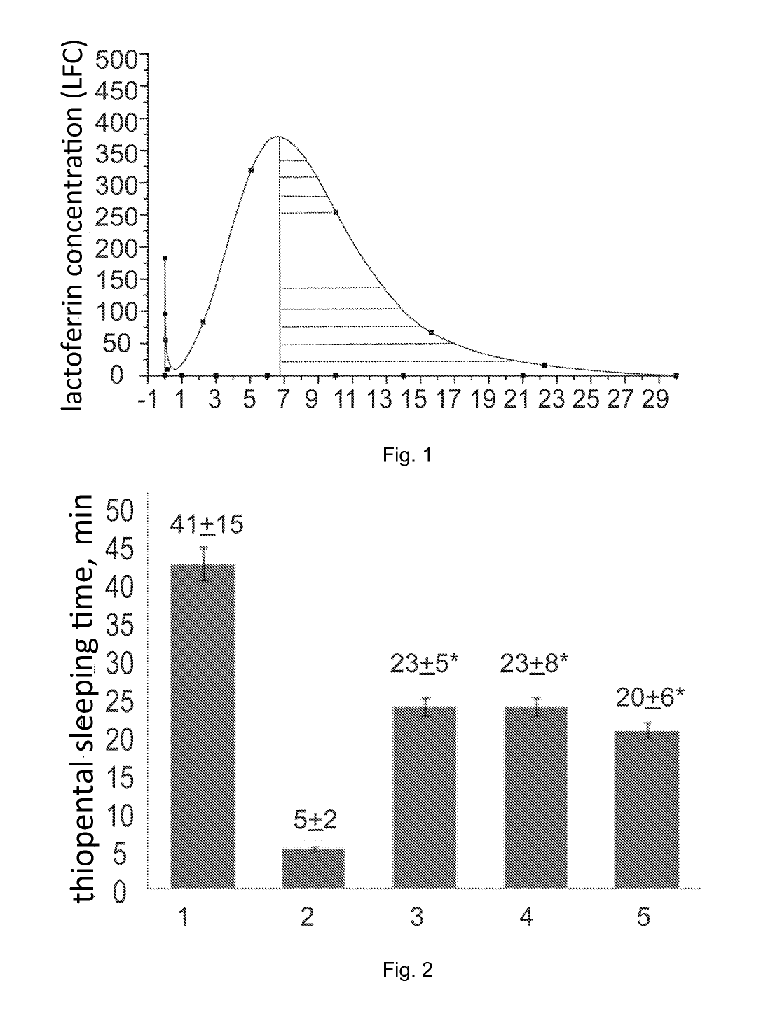

FIG. 1 shows the pharmacokinetic curve characterizing the concentration of human lactoferrin in the serum of rats after a single intravenous dose of a pharmaceutical composition of non-replicating nanoparticles 4.3×10¹¹ v.p./m² and native lactoferrin 10 mg/kg.

Analysis of data represented in the figure showed that the concentration of lactoferrin increases in form of two peaks. First ascent begins with the introduction of a pharmaceutical composition and reaches a peak after 17 minutes with a maximum concentration Cₘₐₓ=140 mkg/ml, and then begins to fall down to the 12^{th} hour after its administration (this segment of the curve reflects the dynamics of native lactoferrin in the composition). However, the fall of concentration of lactoferrin in serum to zero does not occur, since starting from the 12^{th} hour after administration of the composition the second rise is observed, which is due to the start of developing of recombinant lactoferrin by non-replicating nanoparticles, with a peak on 6.8^{th} day and Cₘₐₓ=364 mkg/ml. Next there is a gradual decrease in the concentration and lactoferrin disappears completely from the blood by the day 30^{th}.

Thus, the concentration of human lactoferrin after a single intravenous administration in serum of experimental rats is continuous from the moment of administration and up to 28-30 days, with two peaks of rise in concentration of lactoferrin.

At a separate single administration of native lactoferrin in a therapeutic dose of 10 mg/kg, and the drug is formulated with nanoparticles in the buffer at a dose of 4.3×10¹³ v.p./m², as reference drugs, it is found that the first peak of the curve is due to the native lactoferrin which disappears by the end of the first day after administration, the second peak corresponds to the time of expression of recombinant lactoferrin by non-replicating nanoparticles, which begins only with the 12^{th} hour after injection and lasts up to 28-30 days. Thus, united presence of these two drugs in one pharmaceutical composition allows maintaining the concentration of human lactoferrin in the blood beginning from the 17^{th} minute after administration, without fall in concentration of human lactoferrin significant for detoxification therapy.

Thus, the estimation of pharmacokinetic curve allows us to recommend a pharmaceutical composition for the treatment of not only chronic, but also acute toxic states, as therapeutic effect based on the detoxifying properties of human lactoferrin begins with the 17^{th} minute after injection and lasts for 28-30 days.

### Example 5

### Evaluation of Detoxifying Action of the Pharmaceutical Composition

The detoxifying action of the pharmaceutical composition was studied on a model of toxicity in animals induced by carbon tetrachloride (CCL₄).

Toxicosis, which occurs when CCl₄ is administered to a mammal, is caused by the following processes: CCl₄ undergoes metabolic transformation in membranes of the endoplasmic reticulum of liver by the enzyme cytochrome P-450 which leads to the formation of free radical metabolites (of CCl₃ type) formed as the result of breaking of the molecules of CCl₄. In the result of gains in peroxidation of lipid complexes of intracellular membranes the enzyme activity and a number of cell's functions (protein synthesis, B-lipoprotein exchange and drugs' metabolism) are disrupted, destruction of nucleotides is developed, etc. It is believed that the main site of formation of free radical metabolites are endoplasmic reticulum and microsomal cells, which leads to degradation and reduced activity of cytochrome P-450—the key enzyme of the microsomal oxidation system. This reduces the rate of metabolism of endogenous and exogenous compounds and weakens the antitoxic function of liver. Assessment of the detoxifying function of the liver was carried out by using of thiopental test, which allows—by duration of narcotic sleep of animals—to evaluate the rate of metabolism of thiopental, implemented by the monooxygenase system of hepatocytes, dependent on cytochrome P-450.

A single dose of 2 ml/kg of a 75% of oil solution of CCl₄ was subcutaneously (s/c) administered to animals. Simultaneously, experimental mice were injected with pharmaceutical composition—once, intravenously, at a dose of 4.3×10¹¹ v.p./m² and 10 mg/kg—of native human lactoferrin. Control groups were intravenously only once injected with a composition containing only the nanoparticles expressing human lactoferrin at a dose of 4.3×10¹¹ v.p./m² or 0.9% of sodium chloride. Thiopental was administered on the 6^{th} day after administration of CCl₄ intraperitoneally (i/p)—in a single dose of 55 mg/kg—and the experimental animals' sleep duration was recorded as a criterion of evaluating the degree of liver toxicity damage.

The FIG. 2 presents data on duration of thiopental sleep in animals inoculated with CCl₄. The bars indicate duration of sleep in groups of animals treated with:
CCl₄;
0.9% of sodium chloride;
native human lactoferrin;
composition containing only non-replicating nanoparticles with an inset of lactoferrin gene;
pharmaceutical composition.

From the data presented in the figure can be seen that CCl₄ administration causes an increase in sleep duration of animals as compared sleep duration in the control group of animals treated with physiological solution (41+/−15 min and 5+/−2 min, respectively), thus indicating decrease in the rate of metabolism of thiopental in the liver and, respectively, weakening of antitoxic function of the liver.

Accordingly, the presented results show that thiopental sleep duration in the experimental group (20+/−6 min) was significantly less compared to the control group of mice (41+/−15 min), treated with CCl₄, but not received a pharmaceutical composition; which means the existence of detoxifying properties of the claimed pharmaceutical composition. Also, sleep of the animals that received only the native human lactoferrin or only the composition containing non-replicating nanoparticles with inset of lactoferrin gene, was reduced—in comparison with a group of CCl₄—up to 23+/−8 minutes, but at the same time was a little longer in comparison with that of the experimental group, which means that the claimed composition has the best detoxifying properties.

Thus, a single intravenous administration of the pharmaceutical composition during the onset of the acute stage of toxicity—caused by administration of CCl₄—have significant detoxifying effect on the body, which is stronger than a separately administered—in comparable amounts—the native lactoferrin composition and that of non-replicating nanoparticles expressing lactoferrin.

Further on, examples of clinical doses received during the pre-clinical studies and measured per m² of surface area of the body, were extrapolated to the human person, because they are equivalent: an average human body surface area is 1.62 m² (Khabriev R. U., Manual on experimental preclinical study of new pharmacological substances, 2000, p. 98; Guidance for Industry. Estimating the Maximum Safe Starting Dose in Initial Clinical Trials for Therapeutics in Adult Healthy Volunteers, US Department of Health and Human Services, Food and Drug Administration Center for Drug Evaluation and Research (CDER), Pharmacology and Toxicology, USA, 2005, p. 7, 19).

### Example 6

Patient P. was admitted with the clinical picture of acute toxic gastroenteritis. The main symptoms: drooling, vomiting, diarrhea, cramping abdominal pain for several hours. Was accomplished: gastric lavage, administration of diuretics and intravenous saline solution; treatment was complemented by introducing the claimed pharmaceutical composition: once, intravenously in a volume of 3 ml, which corresponds to introduction of non-replicating nanoparticles expressing lactoferrin in a dose equal to 7×10¹¹ v.p. and of native lactoferrin in dose of 50 mg per person. Within an hour of emergency aid salivation and vomiting stopped, pains and diarrhea eased. The patient's condition improved. By the end of the first day after assisting clinical symptoms have gone completely, toxicosis was stopped.

### Example 7

Patient V. was admitted with acute ethanol poisoning. Main clinical symptoms: cold clammy skin, flushing of the face and conjunctiva, decreased body temperature, vomiting, involuntary leakage of urine and feces, pupils were contracted and with an increase in respiratory disorders were expanding, slow breathing, frequent and weak pulse. Along with the standard infusion therapy, the removal of toxic shock by introducing of 3 ml of claimed pharmaceutical composition—once, intravenously, in a volume of 3 ml, this corresponds to the introduction of non-replicating nanoparticles expressing lactoferrin in a dose equal to 7×10¹¹ v.p. and native lactoferrin in dose of 100 mg per person—was performed. The patient's condition after the implementation of anti-toxic therapy has significantly improved: breathing and pulse have restored, also—surface of skin and mucous membranes, as well as pupils. By the end of the first day of treatment clinical symptoms have disappeared completely, general condition of the patient has improved significantly.

### Example 8

Patient S. Was admitted with diagnosis: colon cancer, post-surgical and post-chemotherapeutical treatment condition. Postoperatively, the patient has developed toxic hepatitis. After introducing 3 ml of a claimed pharmaceutical composition: once, intravenously, in a volume of 3 ml, this corresponds to introduction of nanoparticles expressing lactoferrin in a dose equal to 7×10¹¹ ph.par. and native lactoferrin in doze of 50mg per person. The reduction of level of total and direct bilirubin in serum was identified: 105/80 μmol/I>8.4/3.9 μmol/I. Toxic hepatitis was cured.

### INDUSTRIAL APPLICABILITY

Industrial applicability can be proved by the following.

Use of the claimed pharmaceutical composition in the pharmaceutical and clinical practice achieves several technical, medical and economic outcomes:
The claimed pharmaceutical composition is biocompatible with the human body and therapeutically it is highly effective;
The claimed pharmaceutical composition is suitable for use as it is entered only once and after then (starting from the 17^{th} minute after injection and within 28-30 days) durably produces human lactoferrin in the human body, creating a concentration in blood that is ten times higher than normal level and that is required to achieve a stable therapeutic effect;
The use of the claimed pharmaceutical composition is economically justified as just a single injection of the drug provides for rapid and prolonged therapeutic effect;
The use of the claimed pharmaceutical composition provides for reduction of labor costs of medical personnel, of medical instruments, and thus of complexity and costs of treatment, as the native lactoferrin requires frequent administration, which in our case is eliminated by introduction of non-replicating nanoparticles that produce large amounts of lactoferrin directly in human's body after a just single administration;
The use of the claimed pharmaceutical composition provides for reducing of need for native lactoferrin, as donor human milk is scarce.

These examples show that the developed pharmaceutical composition allows, after single intravenous administration, to receive effective anti-toxic action starting from the 17^{th} minute after its administration and lasts for 28-30 days, which allows for treatment of various toxic conditions, particularly acute. Thus, the imposed technical problem was solved.

What is claimed is:

1. A pharmaceutical composition for treatment of acute toxic conditions, the composition comprising a protein of human lactoferrin and non-replicating nanoparticles with inserts of a human lactoferrin gene, and a formulating buffer.

2. The pharmaceutical composition, of claim 1, wherein a dose of the pharmaceutical composition is 3 ml.

3. The pharmaceutical composition, of claim 2, wherein the dose of the pharmaceutical composition comprises:
the human lactoferrin from 50 to 100 mg;
the non-replicating nanoparticles—7×10¹¹;
the formulating buffer—the rest.

4. The pharmaceutical composition, of claim 1, wherein the human lactoferrin is a donor human milk lactoferrin.

5. The pharmaceutical composition, of claim 1, wherein the human lactoferrin is any type of the human lactoferrin.
